(19) United States
(12) Patent Application Publication
(10) Pub. No.: US 2021/0330193 A1
(43) Pub. Date: Oct. 28, 2021

(54) BLOOD PRESSURE MEASUREMENT METHOD AND BLOOD PRESSURE MEASUREMENT DEVICE

(71) Applicant: Jiangyu Kangjian Innovation Medical Technology(Chengdu) Co., Ltd, Chengdu (CN)

(72) Inventors: PING-HAO LIU, New Taipei (TW); ZHI-BIN HUANG, Shenzhen (CN); ZHI-BING XU, Hangzhou (CN)

(21) Appl. No.: 16/993,353

(22) Filed: Aug. 14, 2020

(30) Foreign Application Priority Data
Apr. 23, 2020 (CN) 202010328256.0

(51) Int. Cl. A61B 5/0205; G01P 15/18; G01P 13/00; A61B 5/11; A61B 5/00

(52) U.S. Cl. CPC A61B 5/0205; G01P 15/18; A61B 5/02108; A61B 5/1118; A61B 5/746; G01P 13/00

(57) ABSTRACT

A blood pressure measurement method is applied in a blood pressure measurement device. The blood pressure measurement device acquires a first systolic pressure, a first diastolic blood pressure, and a first pulse wave transmitted by a measurement unit, and acquiring a second pulse wave transmitted by a monitoring unit. The blood pressure measurement device further determines a user's activity state according to the second pulse wave, calculates a second systolic pressure and a second diastolic blood pressure by a multi-parameter calibration algorithm according to a user's activity state, the first systolic pressure, the first diastolic blood pressure, the first pulse wave, and outputs the second systolic pressure and the second diastolic blood pressure.

This application claims priority to Chinese Patent Application No. 202010328256.0 filed on Apr. 23, 2020, the contents of which are incorporated by reference herein.

### FIELD

The subject matter herein generally relates to health, and blood pressure measurement technology.

### BACKGROUND

Blood, pumped by the heart, circulates by blood vessels in the body, thus exerting pressure on the blood vessels. Blood pressure is determined by blood type, heart rate, arterial wall elasticity, arterial resistance, so the body's blood pressure will change with mood, sitting position, activity, body temperature, diet, medication, and other factors. Time of day and sleep also have a certain effect on blood pressure. Generally, blood pressure in the evening is higher than blood pressure in the morning. Blood pressure is lowest at night, and rises rapidly after the morning, having a peak in the morning (6 am to 10 am) and in the afternoon (4 pm to 8 pm). Bad sleep or excessive fatigue will raise blood pressure slightly. Existing techniques for measuring blood pressure do not enable all-weather automatic and constant monitoring of blood pressure fluctuations.

### BRIEF DESCRIPTION OF THE DRAWINGS

Many aspects of the disclosure can be better understood with reference to the following drawings. The components in the drawings are not necessarily drawn to scale, the emphasis instead being placed upon clearly illustrating the principles of the disclosure. Moreover, in the drawings, like reference numerals designate corresponding parts throughout the several views.

FIG. 1 is a block diagram of an embodiment of a blood pressure measurement device.

FIG. 2 is a flowchart of an embodiment of a blood pressure measurement method.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The present disclosure, including the accompanying drawings, is illustrated by way of examples and not by way of limitation. Several definitions that apply throughout this disclosure will now be presented. It should be noted that references to “an” or “one” embodiment in this disclosure are not necessarily to the same embodiment, and such references mean “at least one.”

The term “module”, as used herein, refers to logic embodied in hardware or firmware, or to a collection of software instructions, written in a programming language, such as, Java, C, or assembly. One or more software instructions in the modules can be embedded in firmware, such as in an EPROM. The modules described herein can be implemented as either software and/or hardware modules and can be stored in any type of non-transitory computer-readable medium or another storage device. Some non-limiting examples of non-transitory computer-readable media include CDs, DVDs, BLU-RAY, flash memory, and hard disk drives. The term “comprising” means “including, but not necessarily limited to”; it specifically indicates open-ended inclusion or membership in a so-described combination, group, series, and the like.

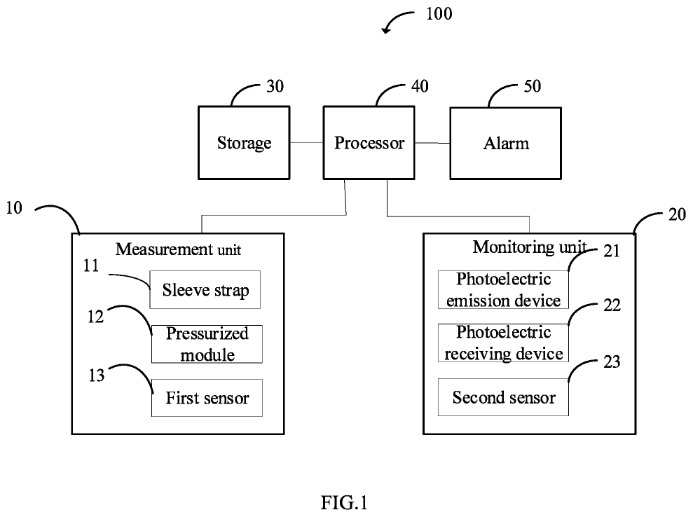

FIG. 1 illustrates a blood pressure measurement device 100. The blood pressure measurement device 100 includes a measurement unit 10, a monitoring unit 20, a storage 30, a processor 40, and an alarm 50. The measurement unit 10 connects to the processor 40. The measurement unit 10 measures a first systolic pressure, a first diastolic blood pressure, and a first pulse wave by an oscilloscope algorithm, and transmits the first systolic pressure, the first diastolic blood pressure, and the first pulse wave to the processor 40. The monitoring unit 20 connects to the processor 40. In one embodiment, the monitoring unit 20 measures a second pulse wave by a photoelectric volume algorithm and transmits the second pulse wave to the processor 40. The storage 30 stores computer program of the blood pressure measurement device 100. The processor 40 executes the computer program to acquire the first systolic pressure, the first diastolic blood pressure, and the first pulse wave transmitted by the measurement unit 10. The processor 40 further acquires the second pulse wave transmitted by the monitoring unit 20, determines a user's activity state according to the second pulse wave, calculates a second systolic pressure and a second diastolic blood pressure by a multi-parameter calibration algorithm according to the first systolic pressure, the first diastolic blood pressure, the first pulse wave, and the second pulse wave. The alarm 50 connects to the processor 40. In one embodiment, the alarm 50 receives an alarm message transmitted by the processor 40, and responses to the alarm message.

In one embodiment, the measurement unit 10 includes a sleeve strap 11, a pressurized module 12, and a first sensor 13. The sleeve strap 11 is set on the user's arm. When measuring the first systolic pressure, the first diastolic blood pressure and the first pulse wave by an oscilloscope algorithm, the measurement unit 10 inflates the sleeve strap 11 by the pressurized module 12, stops inflating the sleeve strap 11 and releases the sleeve strap 11 when a pressure of the pressurized module 12 reaches a preset value. In a process of releasing the sleeve strap 11, the measurement unit 10 measures a change of pulse wave amplitude by the first sensor 13 to get a magnitude change value. When the pulse wave amplitude is in a rising stage of magnitude change value and a ratio between the pulse wave amplitude corresponding to one point of the rising stage and a maximum pulse wave amplitude of the magnitude change value is more than a preset value, the blood pressure measured by the measurement unit 10 is the first systolic pressure. When the pulse wave amplitude is in a decline stage of magnitude change value and a ratio between the pulse wave amplitude corresponding to one point of the rising stage and a maximum pulse wave amplitude of the magnitude change value is less than the preset value, the blood pressure measured by the measurement unit 10 is the first diastolic blood pressure.

In one embodiment, the monitoring unit 20 includes a photoelectric emission device 21, a photoelectric receiving device 22, and a second sensor 23. The photoelectric emission device 21 is controlled by the processor 40 to emit light with preset wavelength to reach to user's skin, and the photoelectric receiving device 22 is controlled by the processor 40 to receive a reflected light with the preset wavelength reflected back from the user's skin, the photoelectric emission device 21 is controlled by the processor 40 to identify a pulsation change of light intensity according to the light intensity of the reflected light, and convert the pulsation change of the light intensity into the second pulse wave. In one embodiment, the second pulse wave is an electrical signal.

In one embodiment, the storage 30 stores data, and computer program of the blood pressure measurement device 100. The processor 40 executes computer program and calls data stored in the storage 30 to realize various functions of the blood pressure measurement device 100. In one embodiment, the storage 30 includes a storage program area and a storage data area, the storage program stores an operating system, at least one function application, etc. The storage data area stores data created by the blood pressure measurement device 100. In one exemplary embodiment, the storage 30 can include various types of non-transitory computer-readable storage mediums. For example, the storage 30 can be an internal storage system of the blood pressure measurement device 100, such as flash memory, a random access memory (RAM) for the temporary storage of information, and/or a read-only memory (ROM) for permanent storage of information. In another embodiment, the storage 30 can also be an external storage system, such as a hard disk, a storage card, or a data storage medium.

In one embodiment, the processor 40 can be a central processing unit, or a common processor, a digital signal processor, a dedicated integrated circuit, ready-made programmable gate arrays or other programmable logic devices, discrete door or transistor logic devices, discrete hardware components, and so on. In another embodiment, the processor 40 can be a microprocessor or any conventional processor. The processor 40 can also be a control center of the blood pressure measurement device 100, using interfaces and lines to connect the parts of the blood pressure measurement device 100.

The processor 40 acquires a first acceleration value in an X-axis of a space cartesian coordinate system, a second acceleration value of a Y-axis of the space cartesian coordinate system, and a third acceleration value of a Z-axis of the space cartesian coordinate system by the second sensor 23 in each unit time of a preset time interval including multiple unit times. The processor 4 fits the first acceleration value, the second acceleration value, and the third acceleration value in each unit time to get a target acceleration value corresponding to the unit time, and adds a number of target acceleration values corresponding to the unit time of the preset time interval including multiple unit times to get a total acceleration value. The processor 40 compares the total acceleration value with a first threshold to get a first comparing result, and compares the total acceleration value with a second threshold to get a second comparing result, determines the user's activity state according to the first comparing result and the second comparing result, where the first threshold is less than the second threshold. In one embodiment, the user's activity state includes a sleep state, a rest state, and a motion state.

In one embodiment, the processor 40 determines the user's activity state is the sleep state when the total acceleration value is less than the first threshold. The processor 40 determines the user's activity state is the rest state when the total acceleration value is more than the first threshold but less than the second threshold. The processor 40 determines the user's activity state is the motion state when the total acceleration value is more than the second threshold.

In one embodiment, the processor 40 calculates a first maximum pulse wave according to the first pulse wave measured by the measuring unit 10, records a peak value of the second pulse wave, a valley value of the second pulse wave and a mean value of the second pulse wave, and calculates an absolute amplitude of the second pulse wave and relative amplitude of the second pulse wave according to the peak value of the second pulse wave, the valley value of the second pulse wave, and the average value of the second pulse wave according to the second pulse wave measured by the monitoring unit. The processor 40 acquires the first systolic pressure and the first diastolic pressure measured by the measuring unit 10, and calculates an unmarked second systolic pressure according to formula

$$BSBP = a \times ESBP + b \times \frac{EMA}{BMA} + c \times PIR + d ,$$

and calculates an unmarked second diastolic pressure according to formula

$$BDBP = e \times EDBP + f \times \frac{EMA}{BMA} + g \times PIR + h ,$$

where BSBP is the marked second systolic pressure, BDBP is the second marked diastolic pressure, ESBP is the first systolic pressure, EDBP is the first diastolic pressure, EMA is the first maximum pulse wave amplitude, BMA is the absolute amplitude of the second pulse wave, PIR is the relative amplitude of the second pulse wave, a, b, c, d, e, f, g, h are coefficients, which are fitted according to the known multiple sets of sample data by regression algorithm. The sample data includes the second systolic pressure, the second diastolic pressure, the first systolic pressure, the first diastolic pressure, the first maximum pulse wave amplitude, the absolute amplitude of the second pulse wave, and the relative amplitude of the second pulse wave. In one embodiment, the processor 40 acquires the sample data, divides the sample data into training sets and validation sets, establishes a regression equation, solve the regression equation to get the coefficients by using the training sets, and verifies the regression equation by using the validation sets.

The processor 40 calculates the unmarked second systolic pressure according to a user's activity state to get the second systolic pressure and calculates the unmarked second diastolic pressure according to activity state to get the second diastolic pressure. In one embodiment, a relationship table includes a number of activity states, a number of first weight values, and a number of second weight values, and defines a relationship between the number of activity states, the number of the first weight values, and the number of second weight values. The processor 40 determines a target first weight value corresponding to the activity state according to the relationship table and multiplies the target first weight value with the unmarked second systolic pressure to get the second systolic pressure. The processor 40 determines a target second weight value corresponding to the activity state according to the relationship table and multiplies the target second weight value with the unmarked second diastolic pressure to get the second diastolic pressure.

In one embodiment, the processor 40 compares the second systolic pressure with a preset systolic pressure range, compares the second diastolic pressure with a preset diastolic pressure range, and when the second systolic pressure is not in the preset systolic pressure range, or the second diastolic pressure is not in the diastolic pressure range, generates a warning message, and sends the warning message to the alarm 50. The alarm 50 outputs the warning message in a form of text or voice. In one embodiment, the alarm 50 can be a voice alarm or a monitor.

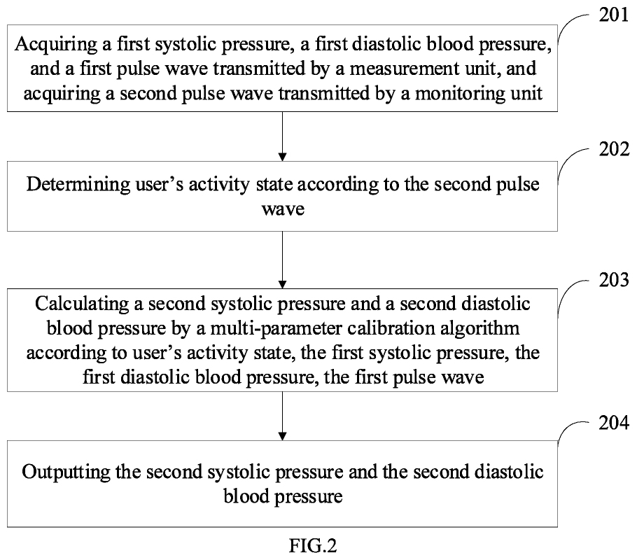

FIG. 2 illustrates a flowchart of an embodiment of a blood pressure measurement method. The blood pressure measurement method is applied in a blood pressure measurement device. The blood pressure measurement method is provided by way of example, as there are a variety of ways to carry out the method. The method described below can be carried out using the configurations illustrated in FIG. 1, for example, and various elements of these figures are referenced in explaining the example method. Each block shown in FIG. 2 represents one or more processes, methods, or subroutines carried out in the example method. Furthermore, the illustrated order of blocks is by example only and the order of the blocks can be changed. Additional blocks may be added or fewer blocks may be utilized, without departing from this disclosure. The example method can begin at block 201.

At block 201, acquiring a first systolic pressure, a first diastolic blood pressure, and a first pulse wave transmitted by a measurement unit, and acquiring a second pulse wave transmitted by a monitoring unit.

At block 202, determining a user's activity state according to the second pulse wave.

At block 203, calculating a second systolic pressure and a second diastolic blood pressure by a multi-parameter calibration algorithm according to a user's activity state, the first systolic pressure, the first diastolic blood pressure, the first pulse wave.

In one embodiment, the blood pressure measurement device calculates a first maximum pulse wave according to the first pulse wave measured by the measuring unit, records a peak value of the second pulse wave, a valley value of the second pulse wave and a mean value of the second pulse wave, and calculates an absolute amplitude of the second pulse wave and relative amplitude of the second pulse wave according to the peak value of the second pulse wave, the valley value of the second pulse wave, and the average value of the second pulse wave according to the second pulse wave measured by the monitoring unit. The blood pressure measurement device acquires the first systolic pressure and the first diastolic pressure measured by the measuring unit, and calculates an unmarked second systolic pressure according to formula

$$BSBP = a \times ESBP + b \times \frac{EMA}{BMA} + c \times PIR + d ,$$

and calculates an unmarked second diastolic pressure according to formula

$$BDBP = e \times EDBP + f \times \frac{EMA}{BMA} + g \times PIR + h ,$$

where BSBP is the marked second systolic pressure, BDBP is the second marked diastolic pressure, ESBP is the first systolic pressure, EDBP is the first diastolic pressure, EMA is the first maximum pulse wave amplitude, BMA is the absolute amplitude of the second pulse wave, PIR is the relative amplitude of the second pulse wave, a, b, c, d, e, f, g, h are coefficients, which are fitted according to the known multiple sets of sample data by a regression algorithm.

In one embodiment, the blood pressure measurement device acquires the sample data, wherein the sample data includes the second systolic pressure, the second diastolic pressure, the first systolic pressure, the first diastolic pressure, the first maximum pulse wave amplitude, the absolute amplitude of the second pulse wave, and the relative amplitude of the second pulse wave. The blood pressure measurement device divides the sample data into training sets and validation sets, establishes a regression equation, solve the regression equation to get the coefficients by using the training sets, and verifies the regression equation by using the validation sets.

In one embodiment, the blood pressure measurement device calculates the unmarked second systolic pressure according to a user's activity state to get the second systolic pressure, and calculates the unmarked second diastolic pressure according to the user's activity state to get the second diastolic pressure. In one embodiment, a relationship table includes a number of the user's activity states, a number of first weight values, and a number of second weight values, and defines a relationship between the number of the user's activity states, the number of the first weight values, and the number of second weight values. The blood pressure measurement device determines a target first weight value corresponding to the user's activity state according to the relationship table and multiplies the target first weight value with the unmarked second systolic pressure to get the second systolic pressure. The blood pressure measurement device determines a target second weight value corresponding to the user's activity state according to the relationship table and multiplies the target second weight value with the unmarked second diastolic pressure to get the second diastolic pressure.

At block 204, outputting the second systolic pressure and the second diastolic blood pressure.

In one embodiment, the method further includes: comparing the second systolic pressure with a preset systolic pressure range, comparing the second diastolic pressure with a preset diastolic pressure range, and when the second systolic pressure is not in the preset systolic pressure range or the second diastolic pressure is not in the diastolic pressure range, generating a warning message, and sending the warning message to an alarm.

It should be emphasized that the above-described embodiments of the present disclosure, including any particular embodiments, are merely possible examples of implementations, set forth for a clear understanding of the principles of the disclosure. Many variations and modifications can be made to the above-described embodiment(s) of the disclosure without departing substantially from the spirit and principles of the disclosure. All such modifications and variations are intended to be included herein within the scope of this disclosure and protected by the following claims.

What is claimed is:

1. A blood pressure measurement device comprising:
a measurement unit configured to measure a first systolic pressure, a first diastolic blood pressure, and a first pulse wave by an oscilloscope algorithm;
a monitoring unit configured to measure a second pulse wave by a photoelectric volume algorithm;
a processor coupled to the measurement unit and the monitoring unit;
a non-transitory storage medium coupled to the processor and configured to store a plurality of instructions, which cause the processor to:
acquire a first systolic pressure, a first diastolic blood pressure, and a first pulse wave transmitted by the measurement unit, and acquire a second pulse wave transmitted by the monitoring unit;
determine a user's activity state according to the second pulse wave;
calculate a second systolic pressure and a second diastolic blood pressure by a multi-parameter calibration algorithm according to the user's activity state, the first systolic pressure, the first diastolic blood pressure, the first pulse wave; and
output the second systolic pressure and the second diastolic blood pressure.

2. The blood pressure measurement device according to claim 1, wherein the monitoring unit comprises a photoelectric emission device and a photoelectric receiving device, the plurality of instructions further cause the processor to:
control the photoelectric emission device to emit light with preset wavelength to reach to the user's skin;
control the photoelectric receiving device to receive a reflected light with the preset wavelength reflected back from the user's skin; and
controls the photoelectric emission device to identify a pulsation change of light intensity according to the light intensity of the reflected light and convert the pulsation change of the light intensity into the second pulse wave.

3. The blood pressure measurement device according to claim 1, wherein the monitoring unit comprises a second sensor, the plurality of instructions further cause the processor to:
acquire a first acceleration value in a X-axis of a space cartesian coordinate system, a second acceleration value of a Y-axis of the space cartesian coordinate system, and the third acceleration value of a Z-axis of the space cartesian coordinate system by the second sensor in each unit time of a preset time interval comprising multiple unit times;
fit the first acceleration value, the second acceleration value, and the third acceleration value in each unit time to get a target acceleration value corresponding to the each unit time, and add a plurality of target acceleration values to get a total acceleration value;
compare the total acceleration value with a first threshold to get a first comparing result, and compare the total acceleration value with a second threshold to get a second comparing result; and
determine the user's activity state according to the first comparing result and the second comparing result, wherein the first threshold is less than the second threshold, and the user's activity state comprises a sleep state, a rest state, and a motion state.

4. The blood pressure measurement device according to claim 1, wherein the plurality of instructions further causes the processor to:
calculate a first maximum pulse wave according to the first pulse wave measured by the measuring unit;
record a peak value of the second pulse wave, a valley value of the second pulse wave and a mean value of the second pulse wave, and calculate an absolute amplitude of the second pulse wave and a relative amplitude of the second pulse wave according to the peak value of the second pulse wave, the valley value of the second pulse wave, and the average value of the second pulse wave according to the second pulse wave measured by the monitoring unit;
acquire the first systolic pressure and the first diastolic pressure measured by the measuring unit, and calculate an unmarked second systolic pressure according to formula
$$BSBP = a \times ESBP + b \times \frac{EMA}{BMA} + c \times PIR + d ,$$
and calculate an unmarked second diastolic pressure according to formula
$$BDBP = e \times EDBP + f \times \frac{EMA}{BMA} + g \times PIR + h ,$$
wherein BSBP is the marked second systolic pressure, BDBP is the second marked diastolic pressure, ESBP is the first systolic pressure, EDBP is the first diastolic pressure, EMA is the first maximum pulse wave amplitude, BMA is the absolute amplitude of the second pulse wave, PIR is the relative amplitude of the second pulse wave, a, b, c, d, e, f, g, h are coefficients, which are fitted according to the known multiple sets of sample data by regression algorithm; and
calculate the unmarked second systolic pressure according to the user's activity state to get the second systolic pressure and calculate the unmarked second diastolic pressure according to the user's activity state to get the second diastolic pressure.

5. The blood pressure measurement device according to claim 4, wherein the plurality of instructions further causes the processor to:
determine a target first weight value corresponding to the user's activity state according to a relationship table, wherein the relationship table comprises a plurality of the user's activity states, a plurality of first weight values, and a plurality of second weight values;
multiply the target first weight value with the unmarked second systolic pressure to get the second systolic pressure; and
determine a target second weight value corresponding to the user's activity state according to the relationship table and multiply the target second weight value with the unmarked second diastolic pressure to get the second diastolic pressure.

6. The blood pressure measurement device according to claim 1, wherein the plurality of instructions further causes the processor to:
compare the second systolic pressure with a preset systolic pressure range, and compare the second diastolic pressure with a preset diastolic pressure range; and
when the second systolic pressure is not in the preset systolic pressure range, or the second diastolic pressure is not in the diastolic pressure range, generate a warning message, and send the warning message.

7. A blood pressure measurement method comprising:
acquiring a first systolic pressure, a first diastolic blood pressure, and a first pulse wave transmitted by a measurement unit, and acquiring a second pulse wave transmitted by a monitoring unit;
determining a user's activity state according to the second pulse wave;
calculating a second systolic pressure and a second diastolic blood pressure by a multi-parameter calibration algorithm according to the user's activity state, the first systolic pressure, the first diastolic blood pressure, the first pulse wave; and
outputting the second systolic pressure and the second diastolic blood pressure.

8. The blood pressure measurement method according to claim 7, further comprising:
controlling a photoelectric emission device to emit light with preset wavelength to reach to the user's skin;
controlling a photoelectric receiving device to receive a reflected light with the preset wavelength reflected back from the user's skin; and
controlling the photoelectric emission device to identify a pulsation change of light intensity according to the light intensity of the reflected light, and converting the pulsation change of the light intensity into the second pulse wave.

9. The blood pressure measurement method according to claim 7, further comprising:
acquiring a first acceleration value in a X-axis of a space cartesian coordinate system, a second acceleration value of a Y-axis of the space cartesian coordinate system, and the third acceleration value of a Z-axis of the space cartesian coordinate system by a second sensor in each unit time of a preset time interval comprising multiple unit times;
fitting the first acceleration value, the second acceleration value, and the third acceleration value in each unit time to get a target acceleration value corresponding to the each unit time, and adding a plurality of target acceleration values to get a total acceleration value;
comparing the total acceleration value with a first threshold to get a first comparing result, and comparing the total acceleration value with a second threshold to get a second comparing result; and
determining the user's activity state according to the first comparing result and the second comparing result, wherein the first threshold is less than the second threshold, and the user's activity state comprises a sleep state, a rest state, and a motion state.

10. The blood pressure measurement method according to claim 7, further comprising:
calculating a first maximum pulse wave according to the first pulse wave measured by the measuring unit;
recording a peak value of the second pulse wave, a valley value of the second pulse wave and a mean value of the second pulse wave, and calculating an absolute amplitude of the second pulse wave and a relative amplitude of the second pulse wave according to the peak value of the second pulse wave, the valley value of the second pulse wave, and the average value of the second pulse wave according to the second pulse wave measured by the monitoring unit;
acquiring the first systolic pressure and the first diastolic pressure measured by the measuring unit, and calculating an unmarked second systolic pressure according to formula
$$BSBP = a \times ESBP + b \times \frac{EMA}{BMA} + c \times PIR + d ,$$
and calculating an unmarked second diastolic pressure according to formula
$$BDBP = e \times EDBP + f \times \frac{EMA}{BMA} + g \times PIR + h ,$$
wherein BSBP is the marked second systolic pressure, BDBP is the second marked diastolic pressure, ESBP is the first systolic pressure, EDBP is the first diastolic pressure, EMA is the first maximum pulse wave amplitude, BMA is the absolute amplitude of the second pulse wave, PIR is the relative amplitude of the second pulse wave, a, b, c, d, e, f, g, h are coefficients, which are fitted according to the known multiple sets of sample data by regression algorithm; and
calculating the unmarked second systolic pressure according to the user's activity state to get the second systolic pressure and calculating the unmarked second diastolic pressure according to the user's activity state to get the second diastolic pressure.

11. The blood pressure measurement method according to claim 10, further comprising:
determining a target first weight value corresponding to the user's activity state according to a relationship table, wherein the relationship table comprises a plurality of the user's activity states, a plurality of first weight values, and a plurality of second weight values;
multiplying the target first weight value with the unmarked second systolic pressure to get the second systolic pressure; and
determining a target second weight value corresponding to the user's activity state according to the relationship table and multiplying the target second weight value with the unmarked second diastolic pressure to get the second diastolic pressure.

12. The blood pressure measurement method according to claim 7, further comprising:
comparing the second systolic pressure with a preset systolic pressure range, and comparing the second diastolic pressure with a preset diastolic pressure range; and
when the second systolic pressure is not in the preset systolic pressure range, or the second diastolic pressure is not in the diastolic pressure range, generating a warning message, and sending the warning message.

13. A non-transitory storage medium having stored thereon instructions that, when executed by at least one processor of a blood pressure measurement device, causes the least one processor to execute instructions of a blood pressure measurement method, the blood pressure measurement method comprising:
acquiring a first systolic pressure, a first diastolic blood pressure, and a first pulse wave transmitted by a measurement unit, and acquiring a second pulse wave transmitted by a monitoring unit;
determining a user's activity state according to the second pulse wave;
calculating a second systolic pressure and a second diastolic blood pressure by a multi-parameter calibration algorithm according to the user's activity state, the first systolic pressure, the first diastolic blood pressure, the first pulse wave; and
outputting the second systolic pressure and the second diastolic blood pressure.

14. The non-transitory storage medium according to claim 13, wherein the blood pressure measurement method further comprising:
controlling a photoelectric emission device to emit light with preset wavelength to reach to the user's skin;
controlling a photoelectric receiving device to receive a reflected light with the preset wavelength reflected back from the user's skin; and
controlling the photoelectric emission device to identify a pulsation change of light intensity according to the light intensity of the reflected light and converting the pulsation change of the light intensity into the second pulse wave.

15. The non-transitory storage medium according to claim 13, wherein the blood pressure measurement method further comprising:
acquiring a first acceleration value in a X-axis of a space cartesian coordinate system, a second acceleration value of a Y-axis of the space cartesian coordinate system, and the third acceleration value of a Z-axis of the space cartesian coordinate system by a second sensor in each unit time of a preset time interval comprising multiple unit times;
fitting the first acceleration value, the second acceleration value, and the third acceleration value in each unit time to get a target acceleration value corresponding to the each unit time, and adding a plurality of target acceleration values to get a total acceleration value;
comparing the total acceleration value with a first threshold to get a first comparing result, and comparing the total acceleration value with a second threshold to get a second comparing result; and
determining the user's activity state according to the first comparing result and the second comparing result, wherein the first threshold is less than the second threshold, and the user's activity state comprises a sleep state, a rest state, and a motion state.

16. The non-transitory storage medium according to claim 13, wherein the blood pressure measurement method further comprising:
calculating a first maximum pulse wave according to the first pulse wave measured by the measuring unit;
recording a peak value of the second pulse wave, a valley value of the second pulse wave and a mean value of the second pulse wave, and calculating an absolute amplitude of the second pulse wave and a relative amplitude of the second pulse wave according to the peak value of the second pulse wave, the valley value of the second pulse wave, and the average value of the second pulse wave according to the second pulse wave measured by the monitoring unit;
acquiring the first systolic pressure and the first diastolic pressure measured by the measuring unit, and calculating an unmarked second systolic pressure according to formula BSBP=a×ESBP+b×EMA/BMA+c×PIR+d, and calculating an unmarked second diastolic pressure according to formula BDBP=e×EDBP+f×EMA/BMA+g×PIR+h, wherein BSBP is the marked second systolic pressure, BDBP is the second marked diastolic pressure, ESBP is the first systolic pressure, EDBP is the first diastolic pressure, EMA is the first maximum pulse wave amplitude, BMA is the absolute amplitude of the second pulse wave, PIR is the relative amplitude of the second pulse wave, a, b, c, d, e, f, g, h are coefficients, which are fitted according to the known multiple sets of sample data by regression algorithm; and
calculating the unmarked second systolic pressure according to the user's activity state to get the second systolic pressure and calculating the unmarked second diastolic pressure according to the user's activity state to get the second diastolic pressure.

17. The non-transitory storage medium according to claim 16, wherein the blood pressure measurement method further comprising:
determining a target first weight value corresponding to the user's activity state according to a relationship table, wherein the relationship table comprises a plurality of the user's activity states, a plurality of first weight values, and a plurality of second weight values;
multiplying the target first weight value with the unmarked second systolic pressure to get the second systolic pressure; and
determining a target second weight value corresponding to the user's activity state according to the relationship table and multiplying the target second weight value with the unmarked second diastolic pressure to get the second diastolic pressure.

18. The non-transitory storage medium according to claim 13, wherein the blood pressure measurement method further comprising:
comparing the second systolic pressure with a preset systolic pressure range, and comparing the second diastolic pressure with a preset diastolic pressure range; and
when the second systolic pressure is not in the preset systolic pressure range, or the second diastolic pressure is not in the diastolic pressure range, generating a warning message, and sending the warning message.
